(19) United States
(12) Patent Application Publication
(10) Pub. No.: US 2011/0245610 A1
(43) Pub. Date: Oct. 6, 2011

(54) ENDOSCOPE HOOD

(76) Inventor: Kouichi TANAKA, Ashigarakami-gun (JP)

(21) Appl. No.: 13/040,011

(22) Filed: Mar. 3, 2011

(30) Foreign Application Priority Data
Mar. 31, 2010 (JP) 2010-081048

(51) Int. Cl. A61B 1/00

(52) U.S. Cl. 600/114

(57) ABSTRACT

An endoscope hood includes: a hood part placed on a forward side of a part of a periphery of the leading end surface of an insertion part of an endoscope or an entirety of the periphery; an abutment surface formed in the hood part on a surface opposite to a central axis of the insertion part, the abutment surface being formed on a plane which obliquely intersects with the central axis; and a pass-through hole which is formed in the hood part, and allows a probe of the endoscope pushed out from a tube passage exit to pass therethrough from a side of the central axis to a side of the abutment surface in the hood part, wherein the abutment surface is pressed against a region to be measured in a state where the probe has passed through a pass-through hole, to thereby fix the probe to the region to be measured in a state where the probe is abutted thereagainst in an axial direction of the probe.

### BACKGROUND OF THE INVENTION

1. Field of the Invention

The presently disclosed subject matter relates to an endoscope hood, and more particularly, to an endoscope hood which is used for fixing a probe of an optical tomographic imaging apparatus, an ultrasonic diagnostic apparatus, and the like to a region to be measured at a leading end of an insertion part of an endoscope.

2. Description of the Related Art

Up to now, diagnostic imaging has been widely carried out. In the diagnostic imaging, an insertion part of an endoscope is placed to be inserted into the body cavities of a blood vessel, a bile duct, a pancreatic duct, a stomach, an esophagus, a large intestine, and the like, and a probe placed at a leading end of the insertion part is used to perform radial scanning, whereby a tomographic image of a living body is created. As an example, in an optical coherent tomography (OCT) apparatus, an elongated optical probe with a built-in optical fiber having a leading end to which an optical lens and an optical mirror are attached is used, a signal beam is emitted in a direction substantially orthogonal to an axial direction (longitudinal direction) of the optical probe, and a return beam thereof is acquired, whereby a tomographic image of a region to be measured in the direction substantially orthogonal to the optical probe is created. Further, in the OCT apparatus, the optical mirror at the leading end of the optical fiber is rotated about the axis of the probe to perform radial scanning, whereby a tomographic image of the region to be measured in a circumferential direction of the axis of the probe is created. It should be noted that, similarly for other measurement apparatuses such as an ultrasonic diagnostic apparatus, there has been known an apparatus in which a probe placed at a leading end of an insertion part of an endoscope is used to acquire data of a region to be measured in a direction orthogonal to an axial direction, whereby a tomographic image and the like are created. Therefore, the presently disclosed subject matter is not limitatively applied to the OCT apparatus, and thus can be applied to a probe of an arbitrary apparatus. In this specification, description is given mainly of the case where the presently disclosed subject matter is applied to the optical probe for OCT.

In the case where a tomographic image of a living body is acquired by the OCT apparatus, it is necessary to prevent blurring due to a body motion during data acquisition. As methods of fixing a probe to a region to be measured, there have been disclosed a method of providing a cap which is detachably attached to an external cylinder (sheath) of an optical probe for OCT, and fixing the probe to the region to be measured by a leading end portion of the cap (Japanese Patent Application Laid-Open No. 2001-87269) and a method of providing a fixing cap at a leading end of an insertion part of an endoscope, and pressing the fixing cap against the region to be measured to observe the region (Japanese Patent Application Laid-Open No. 2000-126114).

### SUMMARY OF THE INVENTION

Incidentally, in the case of using the probe capable of radial scanning as described above, that is, the probe which acquires data of the region to be measured in the direction substantially orthogonal to the axial direction of the probe, in order to enable the probe to follow the movement of the region to be measured caused by a body motion or the like, it is desirable to acquire the data in the state where the probe is fixed to the region to be measured while being abutted thereagainst (pressed thereagainst) in the axial direction. In particular, in the case of using the probe as described above to acquire data of the region to be measured within a predetermined range in the axial direction of the probe, the entire probe or an optical system inside of the external cylinder of the probe is moved in the axial direction, to thereby perform linear scanning. In this case, if the data acquisition is enabled while maintaining the state where the probe is abutted against the region to be measured in the axial direction, within the range of the region to be measured within which the probe is abutted thereagainst, it is possible to enhance the positional accuracy of a data acquisition position in the linear scanning, and to reduce blurring of the data (blurring of a created tomographic image) due to the movement of the region to be measured caused by a body motion or the like.

However, up to now, there has not been proposed a method of fixing the probe to the region to be measured in the state where the probe is abutted thereagainst in the axial direction. In addition, in the method disclosed in Japanese Patent Application Laid-Open No. 2001-87269, only the leading end of the cap is fixed to the region to be measured, and hence a wide range cannot be fixed in the axial direction. In addition, in the method disclosed in Japanese Patent Application Laid-Open No. 2000-126114, only the leading end (plain surface) of the probe is fixed, and hence similarly, a wide range cannot be fixed in the axial direction. Therefore, there is a problem that the linear scanning cannot be performed on the region to be measured in a sufficient range.

The presently disclosed subject matter has been made in view of the above-mentioned circumstances, and therefore has an object to provide an endoscope hood which can prevent, in the case of using a probe which is placed at a leading end of an insertion part of an endoscope so as to protrude therefrom and acquires data of a region to be measured in a direction substantially orthogonal to an axial direction, blurring of the data due to the movement of the region to be measured caused by a body motion or the like, and also can realize, in this case, highly accurate linear scanning in a wide range in the axial direction of the probe.

In order to achieve the above-mentioned object, a first aspect of the presently disclosed subject matter provides an endoscope hood which is placed at a leading end of an insertion part of an endoscope, the insertion part having a leading end surface provided with a tube passage exit from which an elongated probe that has passed through a tube passage inside of the insertion part is pushed out, the probe acquiring data of a region to be measured in a direction substantially orthogonal to an axial direction of the probe, the endoscope hood including: a hood part placed on a forward side of a part of a periphery of the leading end surface of the insertion part or an entirety of the periphery; an abutment surface formed in the hood part on a surface opposite to a central axis of the insertion part, the abutment surface being formed on a plane which obliquely intersects with the central axis; and a pass-through hole which is formed in the hood part, and allows the probe pushed out from the tube passage exit to pass therethrough from a side of the central axis to a side of the abutment surface in the hood part, wherein the abutment surface is pressed against the region to be measured in a state where the probe has passed through the pass-through hole, to thereby fix the probe to the region to be measured in a state where the probe is abutted thereagainst in the axial direction.

According to the first aspect, at the time of data acquisition by the probe, the probe pushed out from the tube passage exit at the leading end of the insertion part is caused to pass through the pass-through hole of the hood part to be placed on the abutment surface side, and the abutment surface is pressed against the region to be measured, which makes it possible to fix the probe to the region to be measured in the state where the probe is abutted thereagainst in the axial direction.

A second aspect of the presently disclosed subject matter provides an endoscope hood according to the first aspect, which is detachably attached to the leading end of the insertion part.

A third aspect of the presently disclosed subject matter provides an endoscope hood according to the first aspect, which is formed in a leading end part of an over tube.

The subject matter of the second and third aspects each describe a mode of attaching the hood according to the presently disclosed subject matter to the insertion part of the endoscope.

A fourth aspect of the presently disclosed subject matter provides an endoscope hood according to the third aspect, wherein the over tube includes a balloon.

According to the fourth aspect, the insertion part is fixed inside of the body cavity by using the balloon, whereby an operator is not required to apply a force for pressing the abutment surface of the hood against the region to be measured. Therefore, it is possible to reduce a load on the operator, and to eliminate the need for the operator to have an advanced technique.

A fifth aspect of the presently disclosed subject matter provides an endoscope hood according to any one of the first to fourth aspect, wherein the hood part is optically transparent.

According to the fifth aspect, the observation of a subject through an observation window provided on the leading end surface of the insertion part can be performed via the hood.

A sixth aspect of the presently disclosed subject matter provides an endoscope hood according to any one of the first to fifth aspect, wherein the pass-through hole is formed in a vicinity of a position which a central axis of the tube passage at the tube passage exit passes, the tube passage allowing the probe to pass through the inside of the insertion part.

According to the sixth aspect, it is possible to cause the probe pushed out from the tube passage exit at the leading end of the insertion part to directly pass through the pass-through hole of the hood part.

A seventh aspect of the presently disclosed subject matter provides an endoscope hood according to any one of the first to fifth aspect, wherein the pass-through hole is formed on a backward side of a position which a central axis of the tube passage at the tube passage exit passes, the tube passage allowing the probe to pass through the inside of the insertion part, and the endoscope hood further includes a guide member which is provided at the position which the central axis passes on a leading end side of the pass-through hole, and guides the probe to the pass-through hole.

According to the seventh aspect, the pass-through hole is formed on the backward side compared with the sixth aspect, whereby it is possible to make shorter a length in a front-back direction of the hood part. On the other hand, with this configuration, it is not possible to cause the probe pushed out from the tube passage exit at the leading end of the insertion part to directly pass through the pass-through hole of the hood part, but the provision of the guide member makes it possible to cause the probe pushed out from the tube passage exit to pass through the pass-through hole.

An eighth aspect of the presently disclosed subject matter provides an endoscope hood according to any one of the first to seventh aspects, wherein the pass-through hole has a shape in which a hole width in a direction orthogonal to a front-back direction is made narrower toward one point on the leading end side thereof.

According to the eighth aspect, it is possible to fix the probe at the position which the probe passes at the leading end of the pass-through hole, and thus possible to precisely press the probe against the region to be measured via the abutment surface.

A ninth aspect of the presently disclosed subject matter provides an endoscope hood according to any one of the first to eighth aspects, wherein the hood part includes: a cylindrical body which is placed on the forward side of the entirety of the periphery of the leading end surface of the insertion part; and a plate-like body which is formed by cutting out a part of the cylindrical body so as to be integral with the cylindrical body, and has an abutment surface.

The ninth aspect describes one mode of an overall shape of the hood part.

A tenth aspect of the presently disclosed subject matter provides an endoscope hood according to any one of the first to ninth aspects, wherein the tube passage which allows the probe to pass through the inside of the insertion part is a forceps channel.

The tenth aspect describes a mode in which the forceps channel is used as the tube passage which allows the probe to pass through the inside of the insertion part, and this mode is generally employed when the probe is placed at the leading end of the insertion part.

An eleventh aspect of the presently disclosed subject matter provides an endoscope hood according to any one of the first to tenth aspects, wherein the probe is a flexible probe which is elastically deformable.

The eleventh aspect describes a feature of the probe for which the hood according to the presently disclosed subject matter can be effectively used.

A twelfth aspect of the presently disclosed subject matter provides an endoscope hood according to any one of the first to eleventh aspects, wherein the probe is used for data acquisition in one of an optical tomographic imaging apparatus and an ultrasonic diagnostic apparatus.

The twelfth aspect describes a type of the probe for which the hood according to the presently disclosed subject matter can be effectively used.

According to the presently disclosed subject matter, the probe which is placed at the leading end of the insertion part of the endoscope so as to protrude therefrom can be fixed to the region to be measured while being abutted thereagainst in the axial direction. Accordingly, it is possible to prevent blurring of the data due to the movement of the region to be measured caused by a body motion or the like, and also realize highly accurate linear scanning in a wide range in the axial direction of the probe.

### BRIEF DESCRIPTION OF THE DRAWINGS

FIG. 1 is an overall configuration view illustrating an endoscope system;

FIG. 2 is an enlarged cross sectional view illustrating a leading end of an insertion part;

FIGS. 3A, 3B, and 3C are a central cross sectional view, a bottom view, and a front view respectively illustrating a configuration of a hood according to a first embodiment of the presently disclosed subject matter, which is provided at the leading end of the insertion part of the endoscope;

FIG. 4 is a cross sectional view illustrating a state where an abutment surface of the hood according to the first embodiment is pressed against a region to be measured (living body) at the time of data acquisition;

FIGS. 5A, 5B, and 5C are a central cross sectional view, a bottom view, and a front view respectively illustrating a configuration of a hood according to a second embodiment of the presently disclosed subject matter, which is provided at the leading end of the insertion part of the endoscope;

FIGS. 6A, 6B, and 6C are a central cross sectional view, a bottom view, and a front view respectively illustrating a configuration in which the hood according to the first embodiment is formed in an over tube;

FIGS. 7A, 7B, and 7C are a central cross sectional view, a bottom view, and a front view respectively illustrating a configuration in which the hood according to the second embodiment is formed in an over tube; and

FIGS. 8A and 8B are central cross sectional views illustrating a case where a balloon is provided in the over tube of FIGS. 6A to 6C.

### DETAILED DESCRIPTION OF THE PREFERRED EMBODIMENTS

Hereinafter, an endoscope hood according to preferred embodiments of the presently disclosed subject matter is described in detail with reference to the accompanying drawings.

Overall Configuration

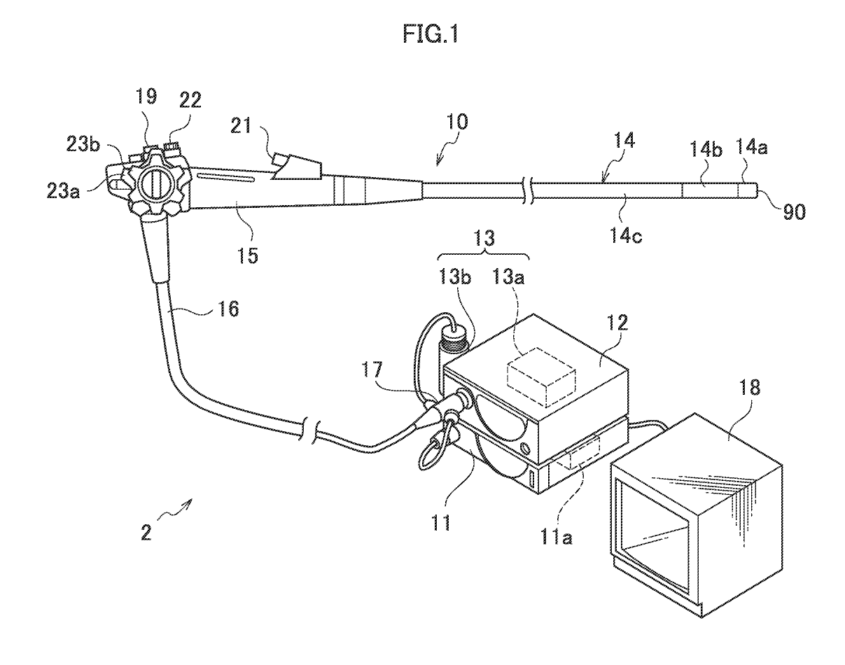

FIG. 1 is an overall configuration view illustrating an endoscope system. An endoscope system 2 illustrated in FIG. 1 includes an endoscope apparatus (endoscope) 10, a processor apparatus 11, a light source apparatus 12 and an air supply/water supply apparatus 13. The air supply/water supply apparatus 13 includes an air supply pump 13a incorporated in the light source apparatus 12 and a washing water tank 13b provided outside of the light source apparatus 12.

The endoscope 10 includes an insertion part 14, an operation part 15, and a universal code 16. The insertion part 14 is a part to be inserted into a body cavity of a patient (body to be examined), and includes a leading end part 14a, a bending part 14b, and a flexible tube part 14c which are connected in the stated order from the leading end side (the opposite side to the operation part 15).

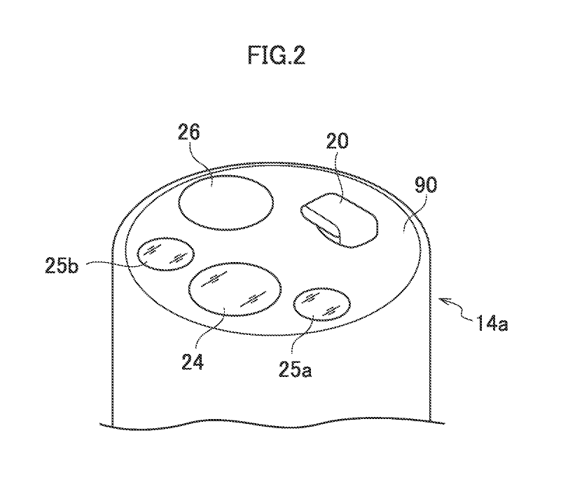

The leading end part 14a is a part including a leading end surface 90 which is a leading end of the insertion part 14, and an image pickup part including image pickup elements such as a CCD (charge-coupled device) and a CMOS (complementary metal-oxide semiconductor) and a lens is provided inside of the leading end part 14a. As illustrated in FIG. 2, an observation window 24, illumination windows 25a and 25b, a forceps exit port 26, and an injection nozzle 20 are provided on the leading end surface 90.

The observation window 24 is formed of an opening formed on the leading end surface 90 and a transparent window member set to the opening, and subject light taken in from the observation window 24 is guided to the image pickup part provided inside of the leading end part 14a, whereby photographing on a front side of the leading end surface 90 is performed.

The illumination windows 25a and 25b are formed of openings on both sides of the observation window 24 and transparent window members set to the respective openings. The illumination light is guided to a light emitting part of the leading end part 14a by a light guide, which passes from the light source apparatus 12 through the insides of the universal code 16, the operation part 15, and the insertion part 14 of the endoscope 10, and then is radiated forward from the illumination windows 25a and 25b, so that the subject in front thereof is illuminated.

The forceps exit port 26 corresponds to an opening portion on the leading end surface 90 side, which is communicated with a forceps port 21 of the operation part 15 via a forceps channel passing through the inside of the insertion part 14, and various treatment tools and a probe inserted from the forceps port 21 are exposed at the forceps exit port 26 in front of the leading end surface 90. In addition, the forceps exit port 26 is also connected to a suction apparatus (not shown) via a suction channel coupled to the forceps channel, and air or washing water injected from the injection nozzle 20, waste matters inside of the body to be examined, and the like are sucked from the forceps exit port 26 into the suction apparatus.

The injection nozzle 20 is attached to an opening formed on the leading end surface 90. The air or the washing water is guided by an air supply/water supply channel which passes from the air supply pump 13a or the washing water tank 13b through the insides of the universal code 16, the operation part 15, and the insertion part 14, and then is injected by the injection nozzle 20 in the direction of the observation window 24, whereby washing of the observation window 24 and the like are performed.

The operation part 15 is a part provided with operation members for allowing an operator to perform various operations, and includes the forceps port 21, an air supply/water supply button 22, a suction button 19, a vertical angle knob 23a and a horizontal angle knob 23b.

As described above, the forceps port 21 is communicated with the forceps exit port 26 via the forceps channel, and the various treatment tools and the probe to be protruded from the forceps exit port 26 are inserted therein.

The air supply/water supply button 22 is an operation button for performing injection start and injection stop of the air or the washing water from the injection nozzle 20 by operating a valve of the air supply/water supply channel. The suction button 19 is an operation button for performing suction start and suction stop from the forceps exit port 26 by operating a valve of the suction channel.

The vertical angle knob 23a and the horizontal angle knob 23b are coupled to a wire insertedly provided in the insertion part 14, and when the knobs 23a and 23b are operated, the wire is pushed or pulled, and the bending part 14b is accordingly bent in a vertical or horizontal direction.

The universal code 16 has one end to which a connector 17 is attached. The connector 17 is a combination connector, and is connected to each of the processor apparatus 11 and the light source apparatus 12. A cable which connects the image pickup part at the leading end of the insertion part 14 with the processor apparatus 11, the light guide which connects the light emitting part at the leading end of the insertion part 14 with the light source apparatus 12, the air supply/water supply channel which connects the injection nozzle 20 at the leading end of the insertion part 14 with the air supply/water supply apparatus 13 (the air supply pump 13a and the washing water tank 13b), and the like are insertedly provided inside of the universal code 16.

The processor apparatus 11 acquires an image pickup signal from the image pickup part (image pickup elements) of the endoscope 10 (leading end part 14a). Then, the processor apparatus 11 performs various types of image processing on the acquired image pickup signal, to thereby create an endoscope image (an image observed from the observation window 24). In addition, the processor apparatus 11 transmits a drive control signal for controlling the drive of the image pickup elements to the image pickup part. The endoscope image created by the processor apparatus 11 is displayed on a monitor 18 connected to the processor apparatus 11 via a cable.

In addition, a controller 11a which is formed of a CPU (central processing unit) and a memory and controls the respective parts of the apparatus is provided inside of the processor apparatus 11. The processor apparatus 11 is connected to the light source apparatus 12 via a communication cable, and the controller 11a communicates various pieces of control information with the light source apparatus 12.

The light source apparatus 12 includes a white color light source, and the light emitted from the light source enters the light guide inside of the endoscope 10 connected thereto via the connector 17. With this configuration, the illumination light is transmitted by the light guide to the light emitting part of the endoscope 10 (leading end part 14a), and then is emitted from the light emitting part via the illumination windows 25a and 25b.

The air supply/water supply apparatus 13 includes the air supply pump 13a incorporated in the light source apparatus 12 and the washing water tank 13b provided outside of the light source apparatus 12. The air supply pump 13a and the washing water tank 13b are connected to the air supply/water supply channel inside of the endoscope 10 via the connector 17. When the air supply/water supply button 22 of the operation part 15 is operated, the injection from the injection nozzle 20 at the leading end of the insertion part 14 can be switched between the air from the air supply pump 13a and the washing water from the washing water tank 13b.

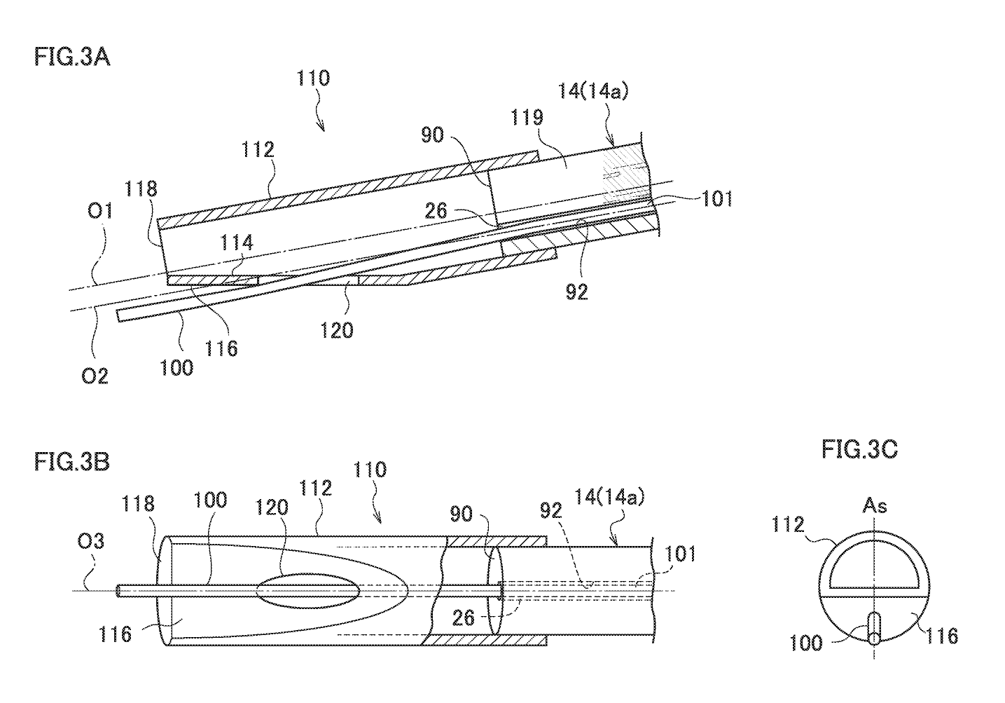

FIGS. 3A, 3B, and 3C are a central cross sectional view, a bottom view, and a front view respectively illustrating a configuration of a hood according to a first embodiment of the presently disclosed subject matter, which is provided at the leading end part 14a of the insertion part 14 (the leading end of the insertion part 14) of the endoscope 10.

A forceps channel 92 which communicates the forceps port 21 of the operation part 15 and the forceps exit port 26 formed on the leading end surface 90 at the leading end of the insertion part 14 is provided inside of the insertion part 14 of the endoscope 10. FIG. 3A illustrates a cross section including a central axis O1 of the insertion part 14 and a central axis O2 of the forceps channel 92 at the forceps exit port 26. A probe 100 of FIG. 3A is inserted from the forceps port 21 of the operation part 15, passes through the forceps channel 92, and is placed so as to protrude forward (toward the far side) from the forceps exit port 26 on the leading end surface 90.

In the present embodiment, the probe 100 is an optical probe for OCT used for acquiring data of a region to be observed (region to be measured) in an optical tomographic imaging apparatus which creates an optical tomographic image of the region to be measured. The probe 100 corresponds to a leading end portion of an elongated probe insertion part 101 which is connected to the optical tomographic imaging apparatus and is inserted from the forceps port 21. In the probe insertion part 101, at least the leading end portion thereof used as the probe 100 is elastically deformable, and the probe insertion part 101 is covered by a transparent flexible external cylinder (sheath). Further, an optical fiber passes through the inside of the probe insertion part 101.

A proximal end of the optical fiber is connected to a connector of a main body (processor) of the optical tomographic imaging apparatus (not illustrated), and the leading end thereof includes, at a portion of the probe 100, an optical system which emits a signal beam from the optical fiber to the region to be measured and takes a return beam from the region to be measured into the optical fiber. This optical system includes, for example, an optical mirror which reflects the signal beam emitted from the optical fiber in the axial direction or the probe 100, and the signal beam is emitted by the optical mirror in the direction substantially orthogonal to the axial direction of the probe 100, so that data of the region to be measured in this direction is acquired. In addition, the optical mirror can rotate inside of the sheath, to thereby rotate an emitting direction of the signal beam in a circumferential direction, which makes it possible to perform radial scanning. Further, the optical system can move in the axial direction of the probe 100 inside of the sheath, to thereby move an emitting position (illuminating position) of the signal beam in the axial direction, which makes it possible to perform linear scanning. At the time of data acquisition, the probe 100 is inserted from the forceps port 21 provided in the operation part 15 of the endoscope 10, passes through the forceps channel 92, and is guided to a position at which the probe 100 protrudes forward from the forceps exit port 26. It should be noted that the presently disclosed subject matter can be adopted as long as the probe 100 is configured to acquire data of the region to be measured in the direction substantially orthogonal to the axial direction, and hence the probe 100 does not necessarily need to be capable of radial scanning, and does not necessarily need to be capable of linear scanning.

On the other hand, in the case where the probe 100 is used to perform measurement, a hood 110 is attached to the leading end of the insertion part 14, and serves to fix the probe 100 to the region to be measured in the state where the probe 100 is abutted thereagainst in the axial direction.

The hood 110 is entirely formed of a member of a transparent material in an integrated manner, and includes a cylindrical part 112 and an abutment part 114. On the basis of a cylindrical shape whose both end surfaces at a proximal end and a leading end are opened (openings 118 and 119), the shape of the cylindrical part 112 is obtained by cutting this cylindrical shape with respect to a plane intersecting, at one point, with the central axis of the cylindrical shape (a plane which is not parallel to the central axis), to thereby remove a part of the leading end. Then, the proximal end at which the opening 119 of the cylindrical part 112 is formed is fitted to an outer circumferential part of the leading end of the insertion part 14 of the endoscope to be detachably fixed to the leading end of the insertion part 14.

The abutment part 114 is a part which is obtained by forming the above-mentioned cut part of the cylindrical part 112 into a flat plate-like shape, and is formed integrally with the cylindrical part 112. It should be noted that at least an outer surface of the abutment part 114 may be formed into a planar shape, and the outer planar surface is referred to as an abutment surface 116. A leading end of the hood 110 has the opening 118 which is surrounded by a leading end edge of the abutment part 114 and a leading end edge of the cylindrical part 112, and a subject located in front of the hood 110 can be observed through the opening 118 from the observation window 24 provided on the leading end surface 90 at the leading end of the endoscope 10. It should be noted that, in the present embodiment, the cylindrical part 112 and the abutment part 114 are formed of the member of a transparent material, and hence the field of view in a range outside of the opening 118 can also be observed via the cylindrical part 112 and the abutment part 114. The leading end of the hood 110 does not necessarily need to have the opening 118, and alternatively may be integrally formed of a plate-like body of a transparent material similarly to the cylindrical part 112 and the abutment part 114. However, a better field of view can be obtained by forming the opening 118. In addition, in the case where the opening 118 is provided, the cylindrical part 112 and the abutment part 114 do not necessarily need to be formed of the member of a transparent material.

In addition, a pass-through hole 120 which allows the probe 100 protruded from the forceps exit port 26 to pass therethrough is formed in the abutment part 114. The pass-through hole 120 is formed at a position through which the probe 100 pushed out from the forceps exit port 26 via the forceps channel 92 passes in the state where the hood 110 is attached to the leading end of the insertion part 14.

That is, in the case as illustrated in FIG. 3C where the hood 110 and the leading end of the insertion part 14 are viewed from the front side (leading end side), the hood 110 has a shape symmetric with respect to an axis of symmetry As, and the axis of symmetry As passes through the center of the cylindrical part 112. On the other hand, on the leading end surface 90 at the leading end of the insertion part 14, the forceps exit port 26 (the central axis O2 of the forceps channel at the forceps exit port 26) is normally located off the center of the leading end surface 90 (the central axis O1 of the insertion part 14). When the hood 110 is attached to the leading end of the insertion part 14, the hood 110 is attached thereto so that the axis of symmetry As thereof intersects with the central axes O1 and O2, and is included in a plane including the central axis O1 and the central axis O2. This plane is orthogonal to the abutment surface 116, and coincides with the cross section of FIG. 3A.

In this state, the pass-through hole 120 of the abutment part 114 is formed into an elliptical shape whose leading end is located in the vicinity of a position at which the central axis O2 of the forceps channel intersects with the abutment part 114 of the hood 110. As illustrated in FIG. 3B, a long axis of the pass-through hole 120 having the elliptical shape coincides with the direction of a central axis O3 in a front-back direction of the abutment surface 116, and the central axis O3 is included in the plane including the central axis O1 and the central axis O2 (the cross section of FIG. 3A).

According to the hood 110, when the probe 100 is inserted from the forceps port 21 of the operation part 15, passes through the forceps channel 92, and is pushed out from the forceps exit port 26, the leading end of the probe 100 travels inside of the hood 110 and abuts against a peripheral edge surface on the leading end side of the pass-through hole 120 of the abutment part 114. After that, when the probe 100 is further pushed out from the forceps exit port 26, the probe 100 is elastically deformed, and the leading end of the probe 100 travels toward the abutment surface 116 (toward the outside of the hood 110) and passes through the pass-through hole 120. Then, the probe 100 is still further pushed out from the forceps exit port 26, so that the probe 100 which has passed through the pass-through hole 120 is placed outside of the abutment surface 116.

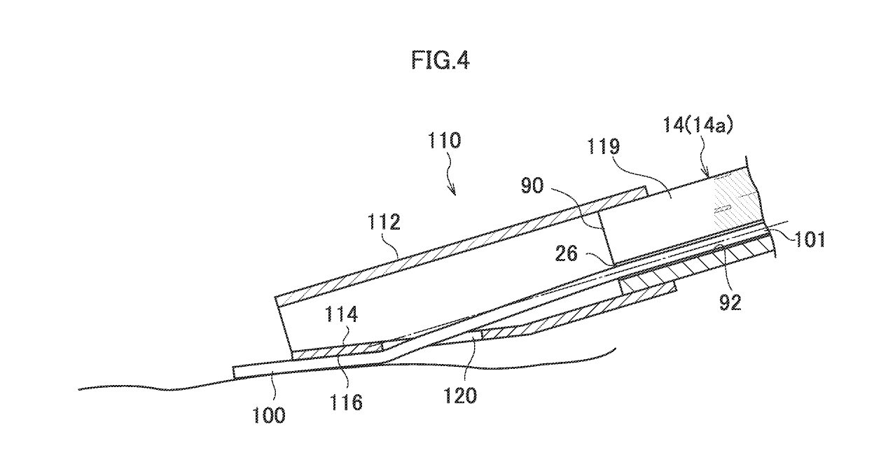

In addition, the probe 100 is urged by its elastic deformation when passing through the pass-through hole 120, in a direction in which the probe 100 abuts against the abutment surface 116 (the upward direction in FIG. 3A), and thus becomes most stable in the state where the probe 100 has passed through the leading end of the pass-through hole 120. The probe 100 is placed at a position along a plane which includes the central axis O3 of the abutment surface 116 and is orthogonal to the abutment surface 116 (that is, a plane including the central axes O1, O2, and O3; hereinafter, referred to as an orthogonal plane including the central axis O3 of the abutment surface 116). At the time of data acquisition, the abutment surface 116 is pressed against the region to be measured as illustrated in FIG. 4, whereby the probe 100 is elastically deformed to be sandwiched between the abutment surface 116 and the region to be measured. At this time, the probe 100 is located along the central axis O3 of the abutment surface 116. Therefore, the probe 100 is precisely pressed against the region to be measured at the position of the central axis O3 of the abutment surface 116 (the central position of the abutment surface 116), and also is fixed to the region to be measured while abutting thereagainst in the axial direction (the axial direction of the probe 100).

Here, in the present embodiment, the pass-through hole 120 is formed into the elliptical shape, and hence the pass-through hole 120 functions as a guide groove which guides the probe 100 to the leading end position of the pass-through hole 120. As a result, the probe 100 can be reliably placed at the position along the orthogonal plane including the central axis O3 of the abutment surface 116, and when the abutment surface 116 is pressed against the region to be measured, the probe 100 can be reliably pressed against the region to be measured at the central position of the abutment surface 116. It should be noted that, if the pass-through hole 120 has a shape whose width becomes smaller toward the leading end position thereof like a triangle having an apex corresponding to the leading end position on the central axis O3 of the abutment surface 116, the probe 100 can be reliably placed at the position along the orthogonal plane including the central axis O3 of the abutment surface 116, and can be appropriately pressed against the region to be measured at the central position of the abutment surface 116. In addition, in the present embodiment, the pass-through hole 120 is formed so as to have its leading end position which is located in the vicinity of the position at which the central axis O2 of the forceps channel 92 intersects with the abutment part 114 (so that the peripheral edge surface at the leading end position of the pass-through hole 120 and the central axis O2 intersect with each other), and the probe 100 is caused to pass through the pass-through hole 120 in the elastically deformed state. Alternatively, the pass-through hole 120 (a hole which is formed so that the central axis O2 comes into no contact with the abutment part 114) is opened at a position which the central axis O2 of the abutment part 114 passes, and the probe 100 is caused to pass through the pass-through hole 120 without being elastically deformed. Also in this way, if the shape of the pass-through hole 120 is limited so as to fall within a range of the vicinity of the central axis O3, the probe 100 can be pressed against the region to be measured at the central position of the abutment surface 116.

Description is given of an operation when the hood 110 is attached to the insertion part 14 as described above and the probe 100 is used to acquire data of the region to be measured inside of the cavity of the body to be examined.

First, as illustrated in FIGS. 3A to 3C, the hood 110 is attached to the leading end of the insertion part 14. Then, with the probe 100 being placed so as not to protrude from the leading end of the insertion part 14, the insertion part 14 is inserted into the cavity of the body to be examined. Then, when the leading end of the insertion part 14 reaches the region to be measured, the probe 100 is inserted from the forceps port 21, is pushed out from the forceps exit port 26 at the leading end of the insertion part 14 as illustrated in FIGS. 3A to 3C to pass through the pass-through hole 120 of the hood 110, and is placed outside of the abutment surface 116 as illustrated in FIGS. 3A to 3C.

Subsequently, as illustrated in FIG. 4, the abutment surface 116 is pressed against the region to be measured, so that the probe 100 is pressed against the region to be measured via the abutment surface 116. As a result, the probe 100 is fixed to the region to be measured while abutting thereagainst in the axial direction, and the data acquisition of the region to be measured in the direction substantially orthogonal to the probe 100 becomes possible.

After that, the data acquisition of the region to be measured using the probe 100 is started. At this time, because the probe 100 is fixed to the region to be measured while abutting thereagainst in the axial direction, even when a position of the region to be measured changes due to a body motion, the probe 100 follows this change. Accordingly, a distance between the probe 100 and a surface of the region to be measured is kept constant, so that the data acquisition is performed with accuracy.

In addition, in the case as in the present embodiment where the probe 100 is an optical probe for OCT, even when the optical system which is provided inside of the sheath and emits a signal beam is moved in the axial direction, to thereby perform scanning (linear scanning) on a scan region within a predetermined range in the axial direction, the leading end of the insertion part 14 and the probe 100 can be kept fixed to the region to be measured during the scanning. Therefore, a created optical tomographic image is not blurred (disturbed), and it is possible to perform the linear scanning with a high positional accuracy of the data acquisition position. Instead of moving the optical system inside of the sheath in the axial direction to thereby move the measurement position in the axial direction, in the case of moving the entire probe 100 (together with the sheath) to thereby perform the linear scanning, the probe 100 is pushed and pulled with the abutment surface 116 being pressed against the region to be measured, whereby the measurement position can be moved in the axial direction. Therefore, it is similarly possible to perform the linear scanning with a high positional accuracy of the data acquisition position. In addition, for a possible range of the linear scanning, as a length in the central axis O3 direction of the abutment surface 116 (a length in the front-back direction thereof) becomes longer, a range in which the linear scanning can be performed with the abutment surface 116 being abutted against the region to be measured becomes larger.

It should be noted that the hood 110 may be formed of a soft material such as silicone rubber and urethane rubber, and may be formed of a hard material such as PC (polycarbonate).

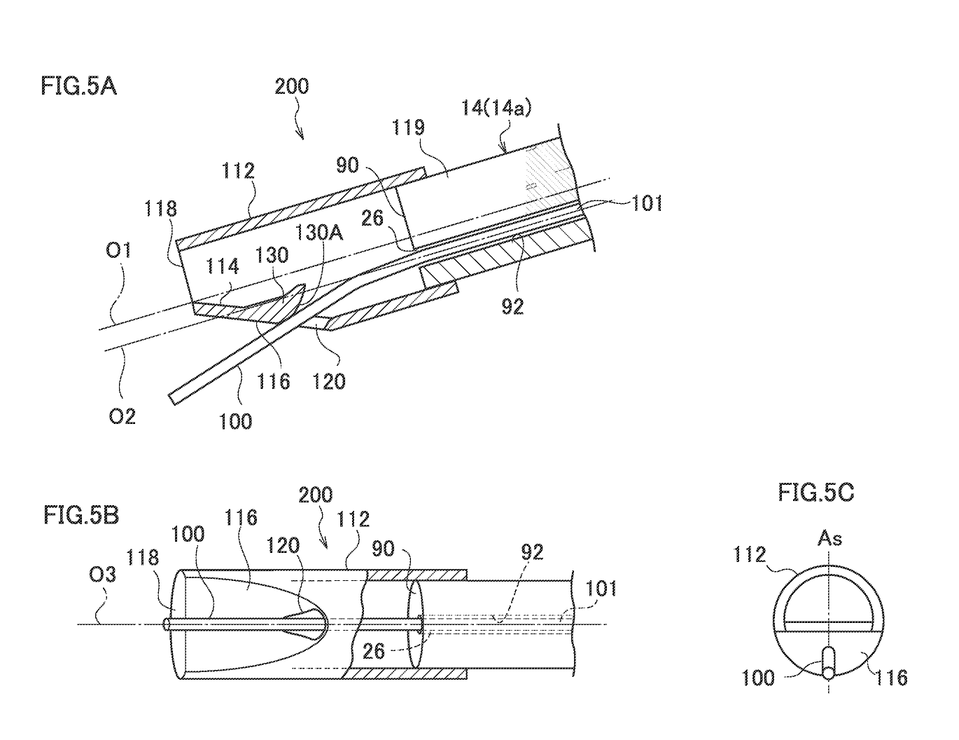

Next, a configuration of a hood according to a second embodiment of the presently disclosed subject matter is illustrated in a cross sectional view, a bottom view, and a front view of FIGS. 5A, 5B, and 5C. The hood according to the second embodiment of FIGS. 5A to 5C has the same basic configuration as that of the hood according to the first embodiment of FIGS. 3A to 3C. Identical or similar components to those of the hood according to the first embodiment are designated by the same reference numeral or character, and only differences from the hood according to the first embodiment are described.

In a hood 200 of FIGS. 5A to 5C, in order to make a total length shorter, the length in the front-back direction (central axis O3 direction) of the abutment part 114 (abutment surface 116) is made shorter. Compared with the first embodiment, the length in the front-back direction of the abutment part 114 is shorter, and an angle formed between the abutment part 114 and the central axis O1 of the insertion part 14 is more acute. On the other hand, the pass-through hole 120 is located closer to the proximal end side, and an effective length of the abutment surface 116 for pressing the probe 100 which has passed through the pass-through hole 120 against the region to be measured, that is, a length between the leading end position of the pass-through hole 120 and the leading end of the abutment surface 116 is not significantly different from (is nearly equal to) that of the first embodiment. Therefore, the range in which the linear scanning can be performed with the abutment surface 116 being pressed against the region to be measured is similar in length to (is nearly equal to) that of the first embodiment.

In addition, a position at which the central axis O2 of the forceps channel 92 intersects with the abutment part 114 and a position of the pass-through hole 120 are significantly different from each other, and hence even when the probe 100 is pushed out from the forceps exit port 26, the probe 100 cannot be caused to directly pass through the pass-through hole 120. Therefore, a guide member 130 which guides the probe 100 pushed out from the forceps exit port 26 to the pass-through hole 120 is provided at the leading end position of the pass-through hole 120.

The guide member 130 includes a guide surface 130A which is formed integrally with or fixedly adhered as a separate member to the abutment part 114 so as to protrude from the abutment part 114 toward the inside of the hood 200 (toward the central axis O1) and is opposed to the forceps exit port 26. The guide surface 130A is formed so as to intersect with the central axis O2 of the forceps channel 92, and is formed of a surface which intersects with the central axis O2 so as to be inclined in the direction from the pass-through hole 120 toward the leading end surface 90 at the leading end of the insertion part 14 (the direction from the far lower side to the near upper side). That is, the guide surface 130A is formed so that an intersection line between the orthogonal plane including the central axis O3 of the abutment surface 116 and the guide surface 130A forms an acute angle with respect to the central axis O2 on the central axis O1 side and on the leading end surface side. It should be noted that the guide surface 130A in FIG. 5A is formed into a rounded shape, but may be a plane.

With the guide member 130, at the time of data acquisition, when the probe 100 is pushed out from the forceps exit port 26, the probe 100 abuts against the guide surface 130A of the guide member 130. Then, when the probe 100 is further pushed out, the probe 100 is elastically deformed by the guide surface 130A to be guided to the pass-through hole 120, and passes through the pass-through hole 120 to be guided to the outside of the abutment surface 116.

In addition, as illustrated in FIG. 5B, the pass-through hole 120 of the hood 200 has a shape different from that of the pass-through hole 120 of the hood 110 according to the first embodiment illustrated in FIGS. 3A to 3C. Unlike the elliptical shape as in the first embodiment, the shape of the pass-through hole 120 is based on a triangle having an apex corresponding to the leading end position on the central axis O3 of the abutment surface 116, and has rounded apexes (a shape obtained by making narrower a width of the leading end of the elliptical shape). The width around the leading end position is generally narrower than the elliptical shape, so that an effect of restricting the position of the probe 100 is enhanced around the leading end position of the pass-through hole 120.

Further, similarly to the guide surface 130A at the leading end position, a peripheral edge surface around the proximal end position of the pass-through hole 120 is also formed of a surface which is inclined in the direction from the far lower side to the near upper side. It should be noted that this peripheral edge surface may be formed by rounding or chamfering. A portion at which the guide surface 130A and the abutment surface 116 are connected to each other is also rounded or chamfered. These surface shapes enable the probe 100 to be easily guided to the pass-through hole 120, and prevent the probe 100 and the like from being damaged at an edge part of the pass-through hole 120.

It should be noted that the configuration concerning the pass-through hole 120 described above in the second embodiment can also be applied to the first embodiment (as well as embodiments described below).

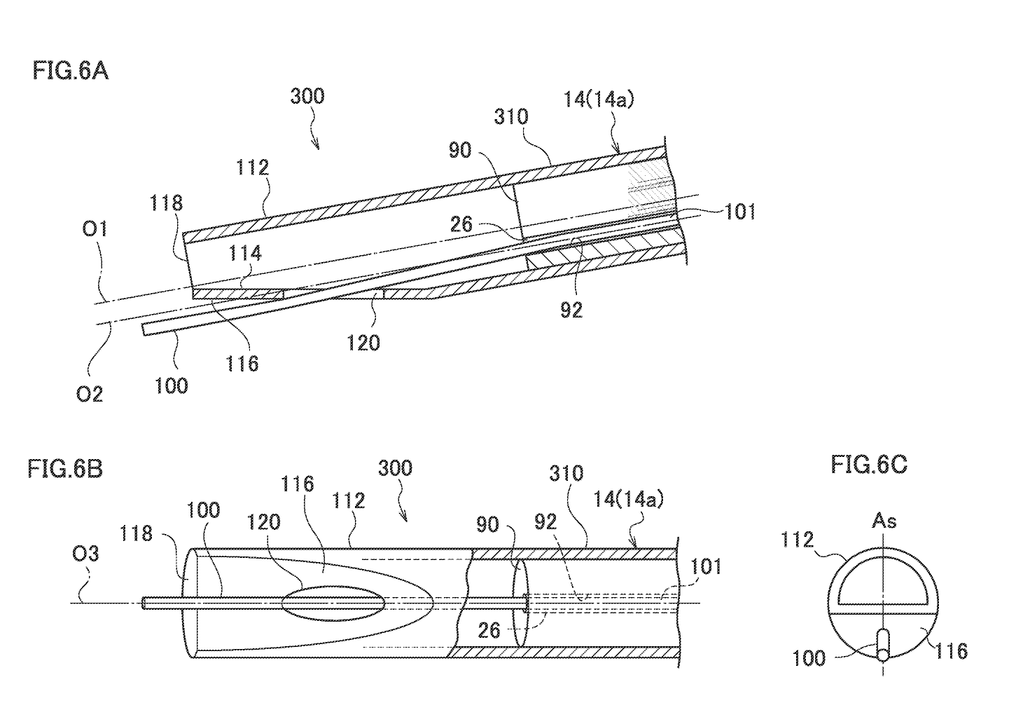

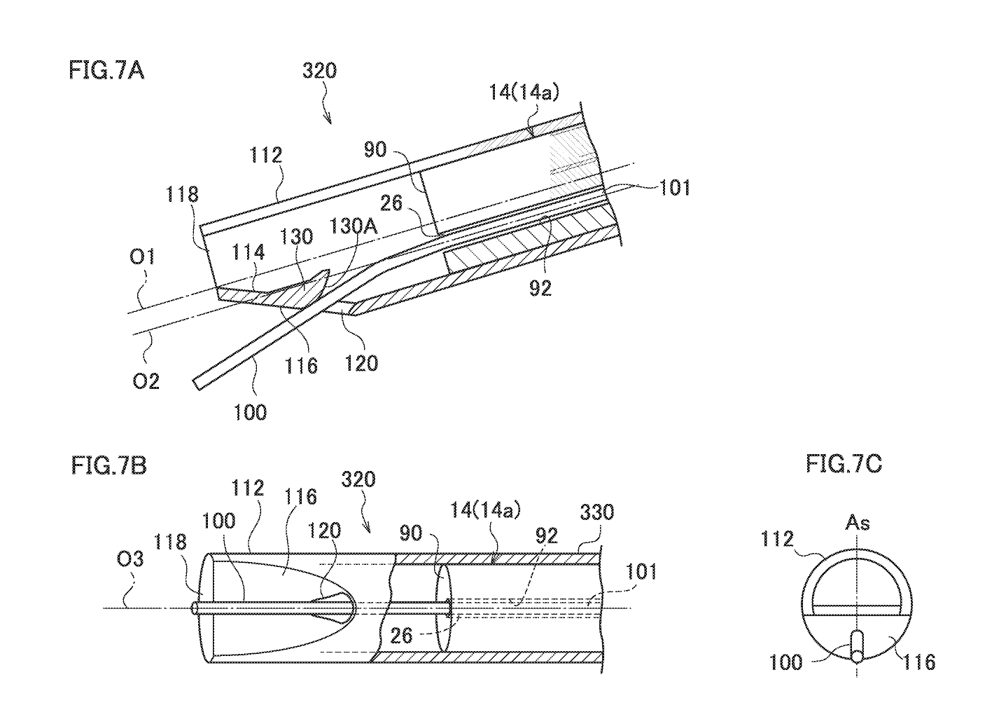

In the hoods 110 and 200 according to the first and second embodiments described above, the proximal end portion of each of the hoods 110 and 200 is fitted and fixed to the outer circumferential part of the leading end of the endoscope 10. Alternatively, a hood such as those according to the first and second embodiments may be formed at a leading end of an over tube which entirely covers the insertion part 14 and guides the insertion part 14 to be inserted into a body cavity. FIGS. 6A, 6B, and 6C illustrate a state where a hood part 300 having the same configuration as that of the hood 110 according to the first embodiment of FIGS. 3A to 3C is formed at a leading end of an over tube 310. FIGS. 7A, 7B, and 7C illustrate a state where a hood part 320 having the same configuration as that of the hood 200 according to the second embodiment of FIGS. 5A to 5C is formed at a leading end of an over tube 330. The configurations of the hood parts 300 and 320 are the same as those of the hoods 110 and 200 according to the first and second embodiments, respectively. Therefore, identical components are designated by the same reference numeral or character, and description thereof is omitted.

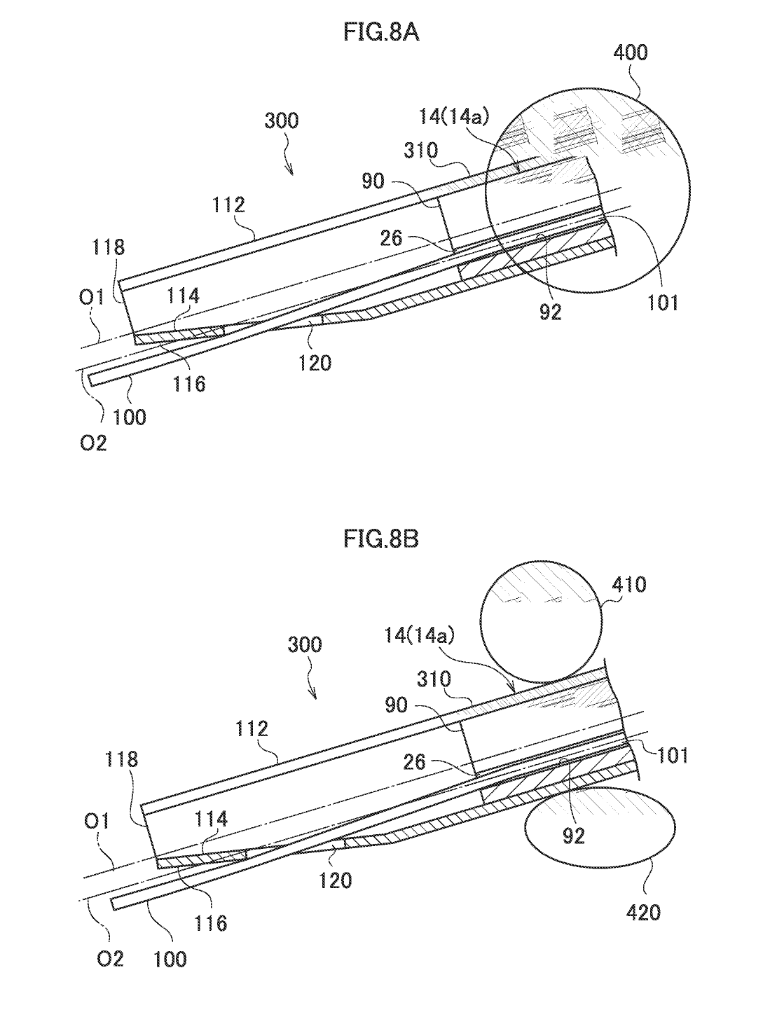

In addition, in the case as illustrated in FIGS. 6A to 6C and FIGS. 7A to 7C where the hood parts 300 and 320 are formed at the leading ends of the over tubes 310 and 330, respectively, it is possible to adopt a configuration including a balloon (balloons) as illustrated in FIGS. 8A and 8B. FIG. 8A illustrates a configuration including one balloon 400 in the embodiment of FIGS. 6A to 6C. FIG. 8B illustrates a configuration including two balloons (double balloon) 410 and 420 in the embodiment of FIGS. 6A to 6C. In the case of using the hoods as illustrated in FIGS. 3A to 3C, FIGS. 5A to 5C, FIGS. 6A to 6C, and FIGS. 7A to 7C, at the time of data acquisition, an operator needs to press the abutment surface of the hood against the region to be measured by a direct operation or an operation of the bending part 14b using the angle knobs 23a and 23b of the operation part 15. On the other hand, in the case of using the balloon(s) as illustrated in FIGS. 8A and 8B, the balloon fills a gap of the body cavity, to thereby enable fixing the leading end of the insertion part 14, and hence a load on the operator is reduced.

In the above-mentioned embodiments, the optical probe for OCT is exemplified as the probe 100, but the presently disclosed subject matter is effective even in a probe of other diagnostic imaging apparatus such as an ultrasonic probe used in an ultrasonic diagnostic apparatus, and is also effective in a probe of a measurement apparatus other than the diagnostic imaging apparatus.

In addition, in the above-mentioned embodiments, for example, as in the hood 110 according to the first embodiment of FIGS. 3A to 3C, a portion other than the abutment part 114 is covered by the cylindrical part 112, but the cylindrical part 112 is not necessarily required. That is, it is not necessary to provide a hood member on the forward side of an entire periphery of the leading end surface 90 of the insertion part 14, and hence the hood member may be provided on the forward side of only a part of the periphery of the leading end surface 90. In particular, this hood member may be provided on the forward side of a part of the periphery of the leading end surface 90 in order to connect the abutment part 114 to a hood member fixed to the outer circumference of the leading end of the insertion part 14.

What is claimed is:

1. An endoscope hood which is placed at a leading end of an insertion part of an endoscope, the insertion part having a leading end surface provided with a tube passage exit from which an elongated probe that has passed through a tube passage inside of the insertion part is pushed out, the probe acquiring data of a region to be measured in a direction substantially orthogonal to an axial direction of the probe, the endoscope hood comprising:
a hood part placed on a forward side of a part of a periphery of the leading end surface of the insertion part or an entirety of the periphery;
an abutment surface formed in the hood part on a surface opposite to a central axis of the insertion part, the abutment surface being formed on a plane which obliquely intersects with the central axis; and
a pass-through hole which is formed in the hood part, and allows the probe pushed out from the tube passage exit to pass therethrough from a side of the central axis to a side of the abutment surface in the hood part,
wherein the abutment surface is pressed against the region to be measured in a state where the probe has passed through the pass-through hole, to thereby fix the probe to the region to be measured in a state where the probe is abutted thereagainst in the axial direction.

2. The endoscope hood according to claim 1, which is detachably attached to the leading end of the insertion part.

3. The endoscope hood according to claim 1, which is formed in a leading end part of an over tube.

4. The endoscope hood according to claim 3, wherein
the over tube includes a balloon.

5. The endoscope hood according to claim 1, wherein
the hood part is optically transparent.

6. The endoscope hood according to claim 1, wherein
the pass-through hole is formed in a vicinity of a position which a central axis of the tube passage at the tube passage exit passes, the tube passage allowing the probe to pass through the inside of the insertion part.

7. The endoscope hood according to claim 1, wherein:
the pass-through hole is formed on a backward side of a position which a central axis of the tube passage at the tube passage exit passes, the tube passage allowing the probe to pass through the inside of the insertion part; and
the endoscope hood further includes a guide member which is provided at the position which the central axis passes on a leading end side of the pass-through hole, and guides the probe to the pass-through hole.

8. The endoscope hood according to claim 1, wherein
the pass-through hole has a shape in which a hole width in a direction orthogonal to a front-back direction is made narrower toward one point on the leading end side thereof.

9. The endoscope hood according to claim 1, wherein
the hood part includes:
a cylindrical body which is placed on the forward side of the entirety of the periphery of the leading end surface of the insertion part; and
a plate-like body which is formed by cutting out a part of the cylindrical body so as to be integral with the cylindrical body, and has an abutment surface.

10. The endoscope hood according to claim 1, wherein
the tube passage which allows the probe to pass through inside of the insertion part is a forceps channel.

11. The endoscope hood according to claim 1, wherein
the probe is a flexible probe which is elastically deformable.

12. The endoscope hood according to claim 1, wherein
the probe is used for data acquisition in one of an optical tomographic imaging apparatus and an ultrasonic diagnostic apparatus.
